(19) United States
(12) Patent Application Publication
(10) Pub. No.: US 2015/0359485 A1
(43) Pub. Date: Dec. 17, 2015

(54) BIOMETRIC SIGNAL CONDUCTION SYSTEM AND METHOD OF MANUFACTURE

(71) Applicant: Mad Apparel, Inc., Redwood City, CA (US)

(72) Inventors: James Artel Berg, Redwood City, CA (US); Gaston MacMillan, Redwood City, CA (US); Hamid Hameed Butt, Redwood City, CA (US); Dhananja Pradhan Jayalath, Redwood City, CA (US); Christopher John Wiebe, Redwood City, CA (US)

(21) Appl. No.: 14/742,420

(22) Filed: Jun. 17, 2015

Related U.S. Application Data
(60) Provisional application No. 62/013,405, filed on Jun. 17, 2014.
(60) Provisional application No. 62/016,373, filed on Jun. 24, 2014.

(51) Int. Cl. A61B 5/00; D05C 13/00; A61B 5/04

(52) U.S. Cl. CPC A61B 5/6804; A61B 5/7203; A61B 5/04; D05C 13/00; A61B 2562/125

(57) ABSTRACT

A system for conducting signals from a set of biosensing contacts and manufacture method thereof, the system comprising: a flexible substrate including a first broad surface and a second broad surface opposing the first broad surface; a set of conductive leads coupled to the first broad surface of the flexible substrate, each of the set of conductive leads including a first region configured to couple to a biosensing contact; a first bonding layer coupled to the first broad surface of the flexible substrate and including a set of openings that expose the first regions of the set of conductive leads for coupling to the set of biosensing contacts; and a second bonding layer coupled to the second broad surface of the flexible substrate and configured to couple the flexible substrate to the garment.

### CROSS-REFERENCE TO RELATED APPLICATIONS

This application claims the benefit of U.S. Provisional Application Ser. No. 62/013,405 filed 17 Jun. 2014, and U.S. Provisional Application Ser. No. 62/016,373 filed 24 Jun. 2014, which are each incorporated in its entirety herein by this reference.

### TECHNICAL FIELD

This invention relates generally to the biometric device field, and more specifically to a new and useful signal conduction system and method of manufacture.

### BACKGROUND

Tracking biometric parameters resulting from periods of physical activity can provide profound insights into improving one's performance and overall health. Historically, users have tracked their exercise behavior by manually maintaining records of aspects of their physical activity, including time points, durations, and/or other metrics (e.g., weight lifted, distance traveled, repetitions, sets, etc.) of their exercise behavior. Exercise tracking systems and software have been recently developed to provide some amount of assistance to a user interested in tracking his/her exercise behavior; however, such systems and methods still suffer from a number of drawbacks. In particular, many systems require a significant amount of effort from the user (e.g., systems rely upon user input prior to and/or after a period of physical activity), capture insufficient data (e.g., pedometers that estimate distance traveled, but provide little insight into an amount of physical exertion of the user), provide irrelevant information to a user, and are incapable of detecting body-responses to physical activity at a resolution sufficient to provide the user with a high degree of body awareness. Other limitations of conventional biometric monitoring devices include one or more of: involvement of single-use electrodes, involvement of electrodes that have limited reusability, involvement of a single electrode targeting a single body location, use of adhesives for electrode placement, electrode configurations that result in user discomfort (e.g., strap-based systems), electrode configurations that are unsuited to motion-intensive activities of the user, and other deficiencies.

Furthermore, integration of biometric tracking systems into garments worn by a user is particularly challenging. Challenges include: coupling conductors to garments in a manner that still allows the garment to move and stretch with motion of the user; preventing sweat (i.e., a conducting fluid from shorting various conductors coupled to a garment); creating an assembly that can be washed and reused without compromising the circuitry and processors through which the system operates; routing signal conduction pathways across seams of a garment; accommodating a high connection density; customizing garment fit to a user; and designing for aesthetics, scalability, and maintaining electrode-skin contact during use by a user.

There is thus a need in the biometric device field to create a new and useful signal conduction system and method of manufacture. This invention provides such a new and useful system and method.

### BRIEF DESCRIPTION OF THE FIGURES

FIG. 1 depicts an embodiment of a system for signal transmission and routing;

FIGS. 2 and 3 depicts an embodiment of a system and supporting elements for signal transmission and routing;

FIGS. 4A and 4B depict variations of a portion of a system for signal transmission and routing;

FIG. 5 depicts a variation of a portion of a system for signal transmission and routing;

FIGS. 6A and 6B depict variations of a portion of a system for signal transmission and routing;

FIGS. 7A and 7B depict variations of stitching patterns in a system for signal transmission and routing;

FIG. 8 depicts an example of a portion of a system for signal transmission and routing;

FIGS. 9A and 9B depict variations and examples of a portion of a system for signal transmission and routing;

FIGS. 10A and 10B depict an embodiment of a method of manufacture for a system for signal transmission and routing; and

FIG. 11 depicts a variation of a portion of a method of manufacture for a system for signal transmission and routing.

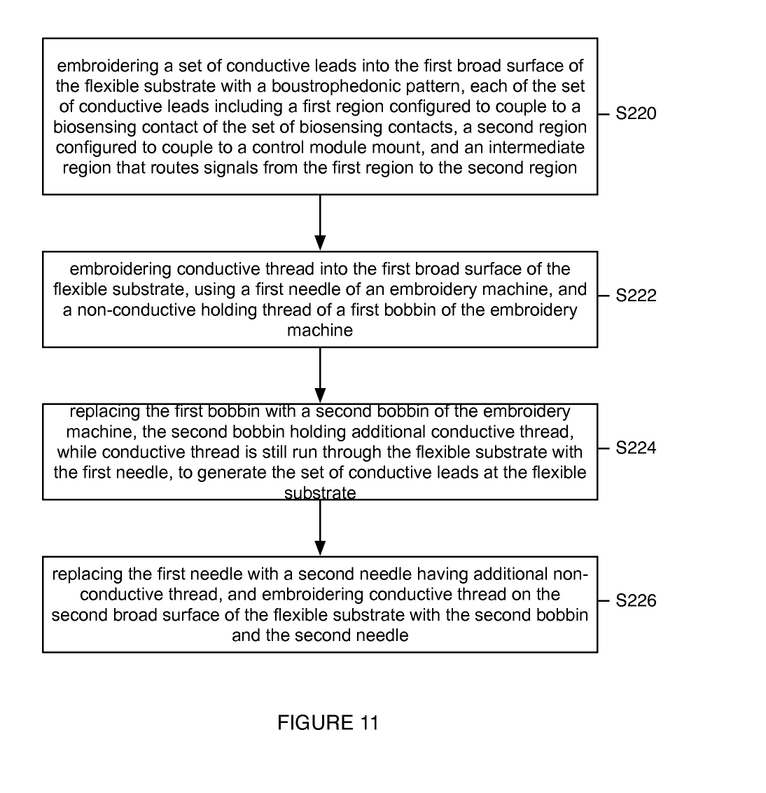

### DESCRIPTION OF THE PREFERRED EMBODIMENTS

The following description of the preferred embodiments of the invention is not intended to limit the invention to these preferred embodiments, but rather to enable any person skilled in the art to make and use this invention.

### 1. System

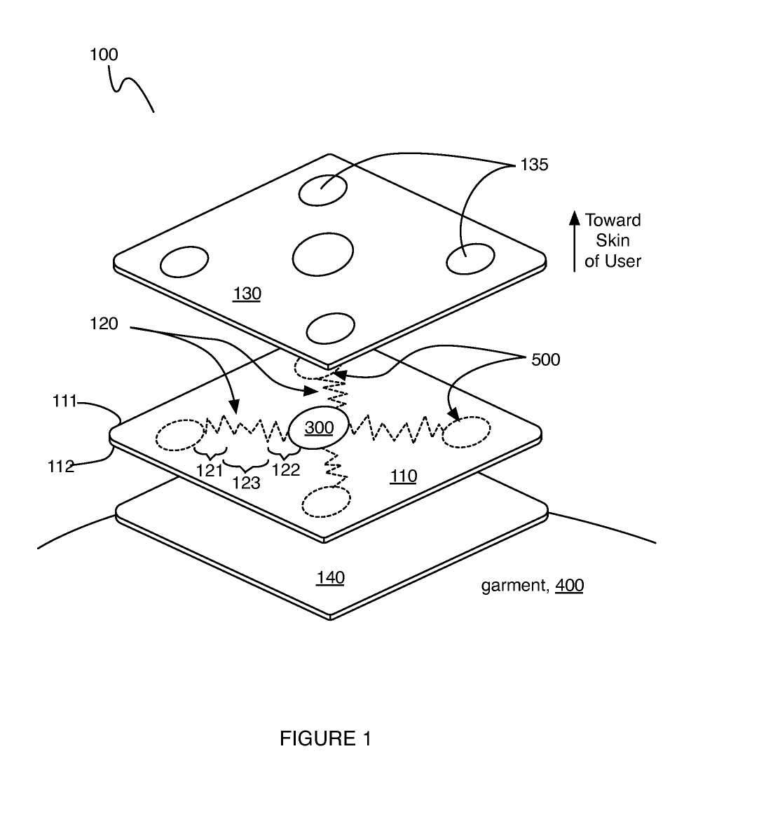

As shown in FIG. 1, an embodiment of a system 100 for conducting signals from a set of biosensing contacts 500 includes: a flexible substrate 110 including a first broad surface 111 and a second broad surface 112 opposing the first broad surface 111; a set of conductive leads 120 coupled to the first broad surface 111 of the flexible substrate 110, each of the set of conductive leads 120 including a first region 121 configured to couple to a biosensing contact, a second region 122 configured to couple to a control module mount 300, and an intermediate region 123 that routes signals from the first region 121 to the second region 122 during use by a user; a first bonding layer 130 coupled to the first broad surface of the flexible substrate 110 and including a set of openings 135 that expose the first regions of the set of conductive leads 120 for coupling to the set of biosensing contacts 500; and a second bonding layer 140 coupled to the second broad surface 112 of the flexible substrate 110 and configured to couple the flexible substrate 110 to a garment 400.

The system 100 functions to facilitate transmission of detected biometric signals from one or more body regions of a user who is performing a type of physical activity, wherein subsequent processing of the detected biometric signals is used to provide information to the user in substantially near real time, such that the user can gain insights into how to maintain or improve performance of the physical activity in a beneficial manner. The system 100 can additionally or alternatively function to protect signal conductor connections, insulate and isolate signal conductors in communication with the system 100, and shield the signal conductor connections from noise sources. Additionally or alternatively, the system 100 can increase the ability of sensors in communication with the system 100 to maintain proper contact with muscles and other sensing sites by providing a stable yet comfortable structure that reliably maintains sensor locations while in use. As such, the system 100 can be used to transfer biometric signals (or other signals) in a manner that has improved wash durability, improved comfort and fit, and improved appearance compared to conventional options.

In variations, the system 100 is configured to facilitate transmission of detected bioelectrical signals generated at multiple body regions of a user who is exercising (e.g., performing aerobic exercise, performing anaerobic exercise), wherein a plurality electrode units in communication with the system 100 can be positioned at multiple body regions of the user, in order to generate a holistic representation of one or more biometric parameters relevant to activity of the user. As such, bioelectrical signals transmittable by the system 100 can include any one or more of: electromyography (EMG) signals, electrocardiography (ECG) signals, electroencephalograph (EEG) signals, galvanic skin response (GSR), bioelectrical impedance (BIA), and any other suitable bioelectrical signal of the user. The system 100 can, however, be configured to transmit any other suitable biosignal data of the user, including one or more of: muscle activity data, heart rate data, movement data, respiration data, location data, skin temperature data, environmental data (e.g., temperature data, light data, etc.), and any other suitable data. Additionally or alternatively, the system 100 can be configured to transmit any other suitable type of electrical signal, including one or more of: audio signals, communication signals, human produced signals, device produced signals, and any other type of signal that can be transferred through a conductive medium.

Preferably, the system 100 is configured to be integrated with a garment 400 worn by a user during a period of physical activity, as described in U.S. application Ser. No. 14/541,446, entitled “System and Method for Monitoring Biometric Signals” and filed on 14 Nov. 2014, U.S. application Ser. No. 14/079,629, entitled “Wearable Architecture and Methods for Performance Monitoring, Analysis, and Feedback” and filed on 13 Nov. 2013, and U.S. application Ser. No. 14/079,621, entitled “Wearable Performance Monitoring, Analysis, and Feedback Systems and Methods” and filed on 30 Jan. 2014, each of which is incorporated herein in its entirety by this reference. As such, the system 100 is preferably configured to provide a liquid-tight interface (e.g., by way of a seal) between conductive components and skin of the user, upon coupling of the system 100 to the user, such that sweat or water cannot penetrate the system 100 and interfere with sensitive portions (e.g., conductive leads) of the system 100 during use. Even further, in relation to integration with a garment 400, the system 100 is preferably configured to be washable (i.e., hand-washable, machine washable, etc.), to be sweat-proof, to be stretchable, to be scalable, to be low-maintenance, and to function properly and in a robust manner in relation to seams of the garment. Furthermore, the system 100 is preferably configured to be designable independent of a particular garment. The system 100 comprises: biometric sensor locations 500 configured to interface with the user's skin during use; a location where a processing system can be connected to the garment 400; and conductors between the two, thereby enabling signal transfer and/or information transfer from the body of the user to a device for processing, storage and/or transmission. In one embodiment, the system 100 is independent of the design lines or seams of the garment 400 and, when bonded to the garment 400, allows signals and/or information to pass across seams. The system 100 can additionally or alternatively allow signals and information to freely route throughout the garment without requiring connections between the individual pieces of the garment joined by seams. As such, the system 100 can provide an improved design for routing signals and biometric information throughout a garment while a user is performing a physical activity.

The system 100 is preferably configured to be used by a user who is away from a research or clinical setting, such that the user is interfacing with a portion of the system 100 while he or she undergoes periods of physical activity in a natural setting (e.g., at a gym, outdoors, etc.). The system 100 can additionally or alternatively be configured to be operated by a user who is in a research setting, a clinical setting, or any other suitable setting. Embodiments, variations, and/or examples of the system 100 can be manufactured according to embodiments, variations, and/or examples of the method 200 described in Section 2 below; however, the system 100 can additionally or alternatively be fabricated using any other suitable method.

### 1.1 System—Supporting Elements

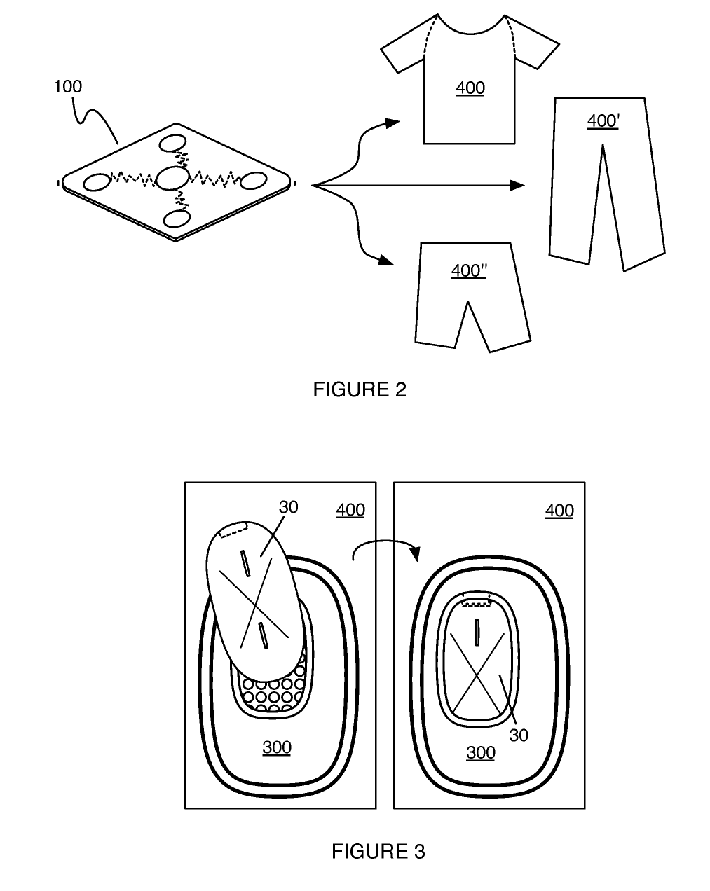

As noted above and as shown in FIG. 2, the system 100 can be integrated with a wearable garment 400, 400′, 400″. The system 100 is preferably further configured to be in communication with a set of biosensing contacts 500 and a portable control module 30 that couples to the garment 400, in operation, by way of a control module mount 300 in direct communication with the system 100. The system 100 is preferably bonded to the garment 400 (e.g., using an adhesive, using a thermal bond, etc.); However, the system 100 can additionally or alternatively provide coupling between electronic components and/or to the garment 400 by way of one or more of: crimp connectors, snap connectors, stitching, a chemical bond, and any other suitable coupling agent.

The garment 400 is preferably composed of a form-fitting and washable material that is configured to be worn on at least a portion of a user's body. In one variation, the system 100 thus couples to the interior of the garment 400 such that the system 100 makes direct physical contact with the skin of the user during use. In other variations, the system 100 can additionally or alternatively be coupled to the exterior of the garment 400, to an inner lining of the garment 400, or directly placed on the user (i.e., without coupling to a garment). Coupling between the system 100 and the garment 400 can be permanent (e.g., by way of heat binding, by way of gluing, by way of stitching, etc.) or non-permanent (e.g., by using Velcro™, by using buttons, by using a light adhesive, etc.). As such, the garment 400 can bias the system 100, coupled to the garment, against skin of the user, when the garment 400 is worn by the user. The garment 400 can thus include a stretchable and/or compressive fabric comprising natural and/or synthetic fibers (e.g., nylon, lycra, polyester, spandex, etc.) to promote coupling (i.e., electrical coupling, mechanical coupling) and/or reduce motion artifacts that could otherwise result from relative motion between the skin of the user and the system 100.

In examples, the garment 400 can include any one or more of: a top (e.g., shirt, jacket, tank top, bra etc.), bottom (e.g., shorts, pants, capris etc.), elbow pad, knee pad, arm sleeve, leg sleeve, socks, undergarment, neck wrap, glove, and any other suitable wearable garment. Furthermore, the garment 400 can include one or more slots, pouches, ports, bases, pathways, channels, cradles, or other features by which the system 100, portable control module 30, the control module mount 300, and/or set of biosensing contacts 100 can permanently or removably couple to the garment 400.

The set of biosensing contacts 500 function to receive signals from the body of the user, and to transmit signals through the system 100 to the portable control module 30 during use by the user. The set of biosensing contacts 500 is preferably an embodiment, variation, or example of the set of biosensing contacts described in U.S. application Ser. No. 14/699,730 entitled “Biometric Electrode System and Method of Manufacture” and filed on 29 Apr. 2015, which is herein incorporated in its entirety by this reference; however, the set of biosensing contacts 500 can additionally or alternatively include any other suitable contacts configured to receive and transmit signals to the system 100.

In relation to the set of biosensing contacts 500, the garment 400 can be configured to position the set of biosensing contacts 500 proximal one or more of: the pectoralis muscles, the abdominal muscles, the oblique muscles, the trapezius muscles, the rhomboid muscles, the teres major muscles, the latissimus dorsi muscles, the deltoid muscles, the biceps muscles, and the triceps muscles when the garment 102 is worn by the user. Additionally or alternatively, the garment 102 can be configured to position the set of biosensing contacts 500 proximal one or more of: the gluteus maximus muscles, the gluteus medius muscles, the vastus lateralis muscles, the gracilis muscles, the semimembranosus muscles, the semitendinosis muscles, the biceps femoris, the quadriceps muscles, the soleus muscles, the gastrocnemius muscles, the rectus femoris muscles, the sartorius muscles, the peroneus longus muscles, and the adductor longus muscles when the garment 102 is worn by the user. Variations of the garment 400 can, however, be configured to position the set of biosensing contacts 500 at the body of the user in any other suitable manner.

As discussed above, the garment 400 can be configured to couple to and/or communicate with one or more portable control modules 30. As such, the combination of the garment 400 and the system 100 can provide one or more sites of coupling with the portable control module(s) 30 in a manner that does not interfere with activity of the user (e.g., during exercise), while allowing the portable control module 30 to interface with all sensor sites governed by the set of biosensing contacts 500. In variations, the portable control module(s) 30 can include circuitry for processing signals, storing data, and/or transmitting data, derived from signals received at the set of biosensing contacts 500 and transmitted through the system 100, to a computing device external to the garment 400. Additionally, the portable control module 30 can cooperate with a control module mount 300 by which the portable control module 30 physically couples to the wearable garment 400 and/or by which the portable control module 30 electrically couples to the system 100. For example, the portable control module 30 can permanently or removably couple to the garment 400 when forming an electrical connection with the system 100, an example of which is shown in FIG. 3. Thus, coupling the portable control module 30 to the garment 400 may include depositing the portable control module 30 into a control module mount 300 coupled to the garment 400 and in communication with a set of conductive leads of the system 100. In one example embodiment, the control module mount 300 includes both physical coupling elements and electrical coupling elements that establish an electrical coupling to the system 100 when the user physically couples the portable control module 30 to the control module mount 300. The portable control module 30 can include embodiments, variations, and examples of the control module described in U.S. application Ser. No. 14/541,446, entitled “System and Method for Monitoring Biometric Signals” and filed on 14 Nov. 2014; however, the portable control module 30 can additionally or alternatively include any other suitable control module.

The system 100 described below can, however, cooperate with or otherwise be integrated with any other suitable elements as described in one or more of: U.S. application Ser. No. 14/541,446, entitled “System and Method for Monitoring Biometric Signals” and filed on 14 Nov. 2014, U.S. application Ser. No. 14/079,629, entitled “Wearable Architecture and Methods for Performance Monitoring, Analysis, and Feedback” and filed on 13 Nov. 2013, and U.S. application Ser. No. 14/079,621, entitled “Wearable Performance Monitoring, Analysis, and Feedback Systems and Methods” and filed on 30 Jan. 2014. Additionally or alternatively, the system 100 can additionally or alternatively be configured to interface with any other suitable element(s).

### 1.2 System—Overview of Information Transfer Inlay

As noted above and as shown in FIG. 1, an embodiment of the system 100 includes: a flexible substrate 110 including a first broad surface 111 and a second broad surface 112 opposing the first broad surface 111; a set of conductive leads 120 coupled to the first broad surface 111 of the flexible substrate 110, each of the set of conductive leads 120 including a first region 121 configured to couple to a biosensing contact, a second region 122 configured to couple to a control module mount 300, and an intermediate region 123 that routes signals from the first region 121 to the second region 122 during use by a user; a first bonding layer 130 coupled to the first broad surface of the flexible substrate 110 and including a set of openings 135 that expose the first regions of the set of conductive leads 120 for coupling to the set of biosensing contacts 500; and a second bonding layer 140 coupled to the second broad surface 112 of the flexible substrate no and configured to couple the flexible substrate no to a garment 400.

The system 100 is preferably manufacturable in a manner that is independent of the garment 400. As such, in one example, the system 100 can be assembled prior to coupling to the garment 400, thus eliminating a requirement for connector elements that maintain electrical connections in the system 100 across different pieces of the garment (e.g., portions of the garment coupled by seams). Thus, in this example, the set of conductive leads 120 of the system 100 can cross seams of the garment 400 without the need to include various complicated design features (e.g., tunnels, connectors, etc.) in the garment 400 that would be prone to reliability issues and breakages and/or cause discomfort to the user. Furthermore, variations of this example of the system 100 can be designed to couple with any type of garment 400 (e.g., shorts, pants, shirts, etc.) by aligning positions of elements of the system 100 relative to a particular garment 400, without the need to change design aspects of the system 100. Furthermore, variations of this example of the system 100 can be designed to couple with any garment material (e.g., cotton, polyester, Spandex, Lycra, Elastane, etc.) without compromising functionality of the system 100. Therefore, the system 100 can provide improved manufacturing scalability and customization with respect to different types of garments 400.

### 1.2.1 System—Substrate

The flexible substrate 110 includes a first broad surface 111 and a second broad surface 112 opposing the first broad surface 111, and functions to facilitate coupling between the set of conductive leads 120 and the set of biosensing contact 500 (described above), and to enable transmission of signals from the set of conductive leads 120 to a portable control module 30 (described above) for downstream processing. The first broad surface 111 is preferably configured to face skin of the user in operation, and the second broad surface 112 is preferably configured to face away from skin of the user and to face an interior surface of the garment 400 in operation; however, the first broad surface 111 and the second broad surface 112 of the flexible substrate no can additionally or alternatively be configured in any other suitable manner. The flexible substrate no is preferably a continuous piece of one or more materials; however, the flexible substrate 110 can alternatively be non-continuous and include disparate regions that are otherwise coupled (e.g., using other elements of the system 100).

While the flexible substrate no is preferably flexible, the flexible substrate no can alternatively comprise regions that are rigid or exhibit both flexibility and rigidity (e.g., by using a combination of rigid and flexible materials). In variations, the flexible substrate no can be composed of one or more of: fabric, cloth, and any other material capable of being stitched together and/or stitched into. In examples, the flexible substrate no can be composed of one or more of: Polyester, Nylon, Polypropylene, wool, Spandex, and any other natural or synthetic material. In one specific example, the flexible substrate no can comprise a nylon-spandex composite (e.g., a nylon-spandex circular knit containing 68% nylon and 32% spandex), which is lightweight and can stretch in multiple directions even upon coupling of the system 100 to the garment 400.

Additionally or alternatively, the flexible substrate 110 can be composed of a polymer composite with conductive elements formed within (e.g., using a printing, thermal forming, molding process, etc.). For example, the flexible substrate no can be formed with a distribution (or pattern) of conductive and/or non-conductive inks that reach a cured state while remaining flexible and stretchable. In this example, the conductive and/or non-conductive inks can be printed onto a first layer of prepared polymer substrate. In some instances a second layer of polymer substrate can be formed onto the first layer of the polymer substrate, thereby sealing and insulating the printed elements within a single multi-layer polymer composite material.

Additionally or alternatively, the flexible substrate no can comprise a material that does not interfere with signal quality and fit of the garment 400. As such, the flexible substrate 110 can additionally or alternatively have anti-static properties to minimize signal interference (e.g., triboelectric effect) that could otherwise result from bending and/or stretching of the flexible substrate 100 or movement between the set of conductive leads 120 and the flexible substrate 110 and/or the system 100 and the garment 400. In one example, the resistance of the anti-static material of the flexible substrate 110 can be selected to not be lower than the input resistance of the circuitry used for acquiring a biometric signal by way of the set of conductive leads 120. As such, the anti-static resistance is configured to prevent formation of spurious current paths that could otherwise reduce the amplitude of the signal as measured at the point of contact at the user's skin, in comparison to the signal received at the input of a portable control module 30 in communication with the system 100.

Additionally or alternatively, in variations where one or more regions of the substrate 110 are rigid, the substrate 110 can comprise of one or more of: a rigid polymer material (e.g., a polytetrafluoroethylene based material), a rigid ceramic material (e.g., FR-4, etc.), a rigid metallic material, or a rigid semiconductor material (e.g., silicon with oxidized regions to define conductive and insulating portions of the substrate). As noted above, composite variations of the substrate 110 can include a combination of materials, isolated to specific regions of the substrate 110, that provide regions of flexibility and regions of rigidity. Additionally or alternatively, materials used in the substrate no can be configured to provide flexibility in certain environmental conditions and rigidity in other environmental conditions.

### 1.2.2 System—Set of Conductive Leads

The set of conductive leads 120 is coupled to the first broad surface 111 of the flexible substrate 110, and is configured to collectively couple to the set of biosensing contacts 500 in operation and configured to enable signal transmission from the set of biosensing contacts 500, through the system 100, and to at least one portable control module 30. As such, the set of conductive leads 120 functions to provide signal routing pathways from the set of biosensing contacts 500, to the portable control module(s)30. As indicated above, the set of conductive leads 120 is preferably coupled to the first broad surface 110 of the flexible substrate 110 configured to face skin of the user, when the garment 400 is worn by the user. Furthermore, upon coupling of the system 100 to the garment 400, at least one of the set of conductive leads 120 preferably crosses a seam of a garment 400 (i.e., in variations wherein the garment 400 has seams). However, the set of conductive leads 120 can alternatively be situated at any other suitable region of the flexible substrate 110, with coupling between the control module mount 300 and the set of biosensing contacts 500 implemented in any other suitable manner. Furthermore, each conductive lead in the set of conductive leads 120 is preferably composed of a metallic material that is electrically conductive; however, the set of conductive leads 120 can additionally or alternatively include any other suitable conductive material (e.g., conductive polymer, etc.).

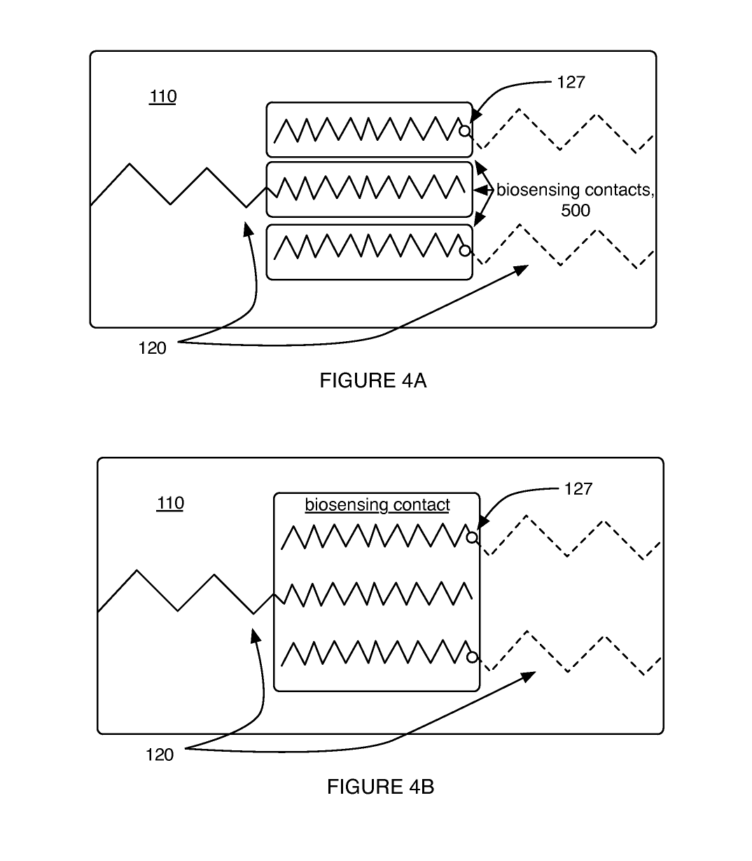

In relation to the set of biosensing contacts 500, the set of conductive leads 120 can be coupled to the set of biosensing contacts in a one-to-one manner, an example of which is shown in FIG. 4A. Alternatively, however, multiple conductive leads can be coupled to a single biosensing contact. In the example shown in FIG. 4B, multiple conductive leads of the set of conductive leads are configured to couple to a single biosensing contact.

The set of conductive leads 120 preferably comprises conductive thread, which can provide one or more conductive paths throughout the system 100 coupled to the garment 400, while not compromising aesthetics or comfort for the user. However, the set of conductive leads 120 can additionally or alternatively comprise conductive wire or any other suitable conductive material having any other suitable form factor.

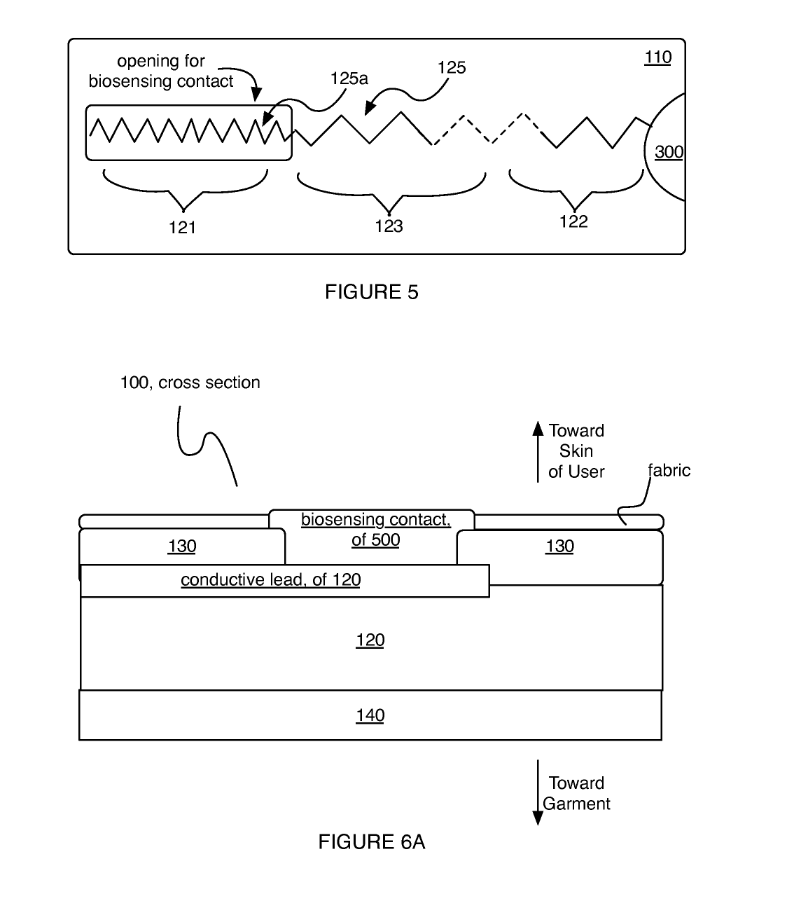

In coupling the set of conductive leads 120 to the flexible substrate 110, one or more of: an embroidery method (e.g., cross-stitching), a conductive epoxy, a crimping method, a soldering method, a laser direct structuring approach, a two-shot molding approach, screen printing approach and any other suitable method can be used to couple the set of conductive leads 120 to the flexible substrate 110. In one variation, as shown in FIG. 5, the set of conductive leads 120 can comprise conductive thread embroidered onto a surface of flexible substrate no, wherein the conductive thread is exposed to enable coupling of the set of biosensing contacts 500 to the conductive thread through openings 135 in the bonding layer(s), as described further below. In a specific example, the conductive thread of the set of conductive leads 120 is a multifilament silver coated nylon core twisted in a 3-ply construction with a resistance per unit length of 5.7 Ω/cm. However, variations of this specific example can implement any other suitable conductive thread and/or one or more of: wire, conductive fabrics, conductive tape, fine conductive wire, printed conductive ink, printed conductive polymer and any other suitable material. Furthermore, one or more conductive leads of the set of conductive leads 120 can have non-uniform conductivity along its length (e.g., by adjusting material composition, by adjusting lead cross section, etc.), thereby enabling manipulation of signal transmission through the conductive lead(s). As such, a conductive lead of the set of conductive leads 120 can have one or more regions of lower conductivity and/or one or more regions of higher conductivity. In one such variation, a region of high conductivity can be used to facilitate signal transmission from the first broad surface 111 to the second broad surface 112 of the flexible substrate no. In similar variations, signal conducting elements of the system 100 can be routed from and/or between broad surfaces of the flexible substrate 110 in any other suitable manner. For instance, two flexible substrate layers, each having conductive traces, can be aligned and coupled together (e.g., facing each other) in order to enable signal conduction within the region between the two flexible substrate layers. Additionally or alternatively, portions of conductive traces between two substrate layers can be electrically coupled (e.g., with a conductive pad, with a conductive adhesive, etc.) in a manner that prevents cross-contact between the conductive traces in an undesired manner. However, signal conducting elements of the system 100 can be routed from and/or between broad surfaces of the flexible substrate no in any other suitable manner

The conductive thread of the set of conductive leads 120 of this variation can have a defined stitching pattern 125, as shown in FIG. 5, that increases surface area contact of the conductive thread with a biosensing contact of the set of biosensing contacts 140. Additionally or alternatively, the stitching pattern can facilitate deformation (e.g., stretching) of the system 100 during use by the user. Preferably, the stitching pattern 125 is boustrophedonic in order to enable stretching and/or contraction during use of the system 100 by the user, without significantly straining the material of the conductive thread. As such, in variations, the stitching pattern 125 can be one or more of: serpentine, zig-zagged, linear, curved, and crossed. However, the conductive thread can additionally or alternatively comprise any other suitable stitching pattern and/or be coupled to the flexible substrate no in any other suitable manner.

As noted above, each of the set of conductive leads 120 preferably includes a first region 121 configured to couple to a biosensing contact of the set of biosensing contacts 500, a second region 122 configured to couple to a control module mount 300, and an intermediate region 123 that routes signals from the first region 121 to the second region 122 during use of the system 100 by a user.

The first region 121 of a conductive lead functions to receive signals from one or more corresponding biosensing contacts, and to transmit received signals through the intermediate region 123 for downstream processing. As such, as noted above and in more detail in relation to the openings of the bonding layer(s) 130, 140, one or more biosensing contacts composed of a conductive material (e.g., conductive silicone, another conductive polymer, etc.) can be coupled to the first region 121 of a conductive lead, thereby forming a continuous electrically conductive interface between the biosensing contact and the first region 121 of the conductive lead. In relation to the first region 121 and an opening of the bonding layer, the configuration of a biosensing contact can be used to compensate for any irregularities in the shape of the opening of the bonding layer, and to form a seal (e.g., waterproof seal, hermetic seal, etc.) to prevent moisture, dust, or other contaminates from penetrating aspects of the system 100 and interfering with signal transmission. As shown in FIG. 5, the first region 121 of the conductive lead preferably has a boustrophedonic pattern 125a that is denser than the stitching pattern 125 of other adjacent portions of the conductive leads, in order to provide more surface area for coupling with the biosensing contact(s). However, the first region 121 can alternatively have a pattern that is not boustrophedonic, and/or is not denser than the stitching pattern 125 of adjacent portions of the conductive leads.

Similar to the first region 121, the second region 122 of a conductive lead functions to receive signals from the intermediate region 123, and to transmit received signals to a portable control module 30, by way of a control module mount 300, for downstream processing. As such, the second region 122 of a conductive lead can terminate at a termination point (e.g., contact region) of a control module mount 300 that couples (e.g., permanently couples, reversibly couples) to a portable control module 30 for signal processing. In particular, the second region 122 preferably has a dedicated position at the control module mount 300, such that signals from a specific body region (governed by the location of the first region 121 of the conductive lead) can be directed to the dedicated position, transmitted through an associated conductor of the control module mount 300, transmitted from the associated conductor to the portable control module 30, and analyzed for provision of insights to the user. The second region 122 of a conductive lead can, however, be configured in any other suitable manner.

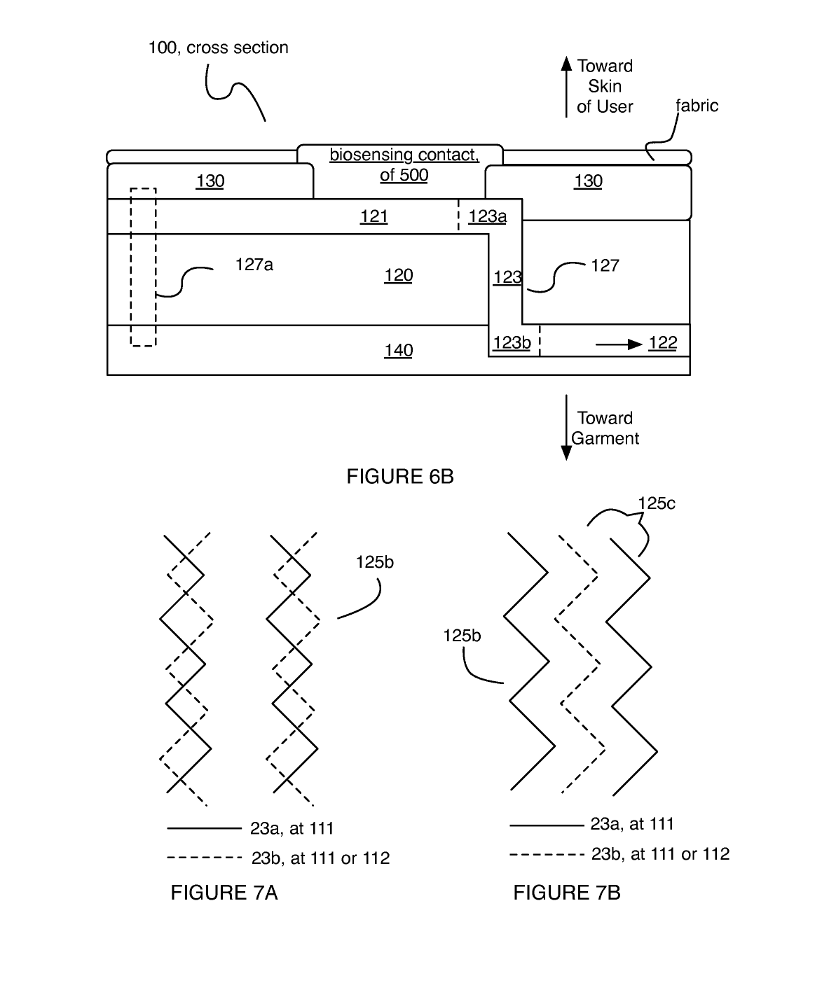

The intermediate region 123 of a conductive lead functions to route signals from the first region 121 to the second region 122 of the conductive lead. The intermediate region 123 of the conductive lead is preferably composed of the same material as the first region 121 and the second region 122 of the conductive lead, and physically contiguous with the first region 121 and the second region 122 of the conductive lead without need for connectors or crimping agents; however, the intermediate region 123 can alternatively comprise a different material composition and/or a different configuration than the first region 121 and/or the second region 122 of the conductive lead. In a first variation, as shown in FIG. 6A, the intermediate region 123 may not pass through the thickness of the flexible substrate 110; however, in a second variation, as shown in FIG. 6B, the intermediate region 123 can pass into the thickness of the flexible substrate 110. As such, in the first variation, the first region 121 and the second region 122 of a conductive lead can be positioned at the same side (e.g., the first broad surface 111, the second broad surface 112) of the flexible substrate 110.

In the second variation, however, the first region 121 can be coupled to the first broad surface 111 and the second region 122 can be coupled to the second broad surface 112, wherein the flexible substrate 110 includes a port 127 through the thickness of the flexible substrate 110 through which the intermediate region 123 passes. The port 127 can be a predefined opening through the thickness of the flexible substrate 110, or can alternatively be generated during manufacturing (e.g., during an embroidery process), as described further in Section 2 below. Additionally or alternatively, the port can include a conductive trace (e.g., a volume of conductive material) to which both the first region 121 and the second region 122 couple in transmitting a signal from a biosensing contact to a portable control module 30. As such, in the second variation, the first region 121 is configured to be positioned between the first broad surface 111 of the flexible substrate 110 and the first bonding layer 130, while the second region 122 is configured to be positioned between the second broad surface 112 of the flexible substrate 110 and the second bonding layer 140.

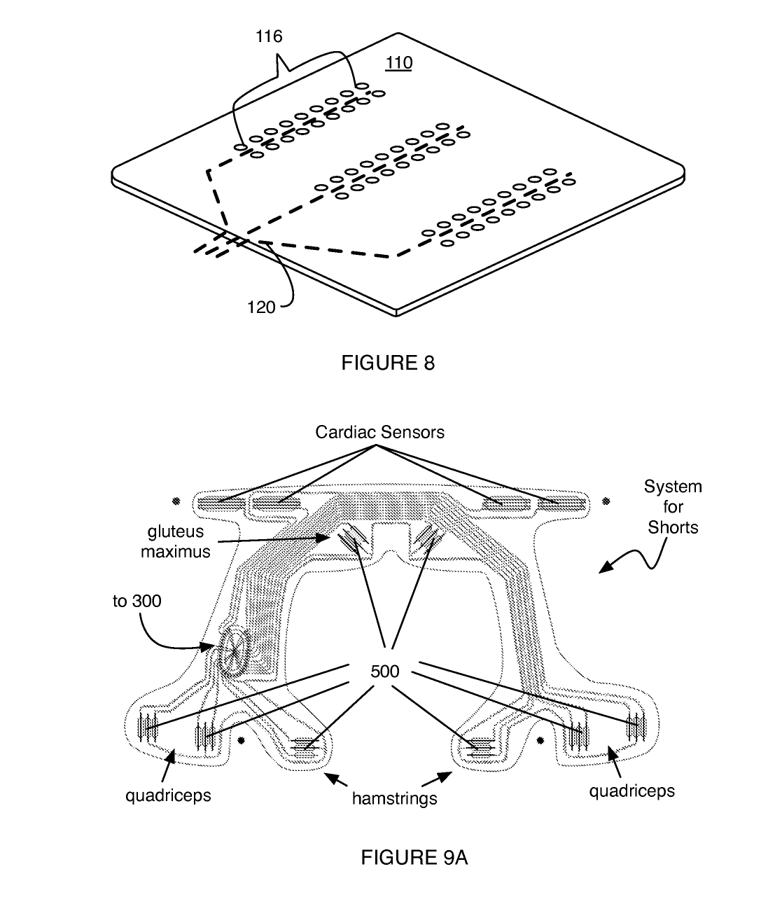

As such, in the second variation, the area of the footprint of the flexible substrate 110 that supports signal transmission can be reduced by routing material of the set of conductive leads 120 on both the first broad surface 111 and the second broad surface 112 of the flexible substrate 110. Reducing the area of the footprint can help to minimize the effect of stretching of the garment 400 during use, once the system 100 is coupled to the garment 400. Also, reducing the area of the footprint reduces the amount of material in the garment making it lighter and more comfortable to the user. Additionally, the configuration of the second variation can allow signal conductors (e.g., portions of the set of conductive leads 120) to overlap without becoming electrically connected. Furthermore, the configuration of the second variation can enable conductive leads associated with paired biosensing contacts (i.e., biosensing contacts from which a differential signal is intended to be extracted) to be routed on opposite sides of the flexible substrate no. Routing conductive leads from paired biosensing contacts on opposite sides of the flexible substrate 110 allows routing the leads in a crossing pattern as shown in FIG. 7A, and described in more detail below.

As shown in FIG. 6B, in the second variation, the intermediate region 123 crosses through the thickness of the flexible substrate no and has a first portion 123a at the first broad surface 111 of the flexible substrate no and a second portion 123b at the second broad surface 112 of the flexible substrate no. In more detail, the intermediate region 123 and port 127 together can form a via, in a manner that is analogous to printed circuit board fabrication (PCB). As described above, in one embodiment the via can pass through the substrate 110 from the first broad surface 111 to the second broad surface 112 (through a via). Additionally or alternatively, the via could pass between intermediate internal layers of the substrate 110 and not be visible from either surface of the substrate (e.g., such that the via is a buried via that is not exposed at a broad surface of the flexible substrate). Additionally or alternatively, the via could pass from a broad surface of the substrate no to an internal layer of the flexible substrate, and only be visible from one broad surface of the flexible substrate (e.g., such that the via is a blind via that is only exposed at one broad surface of the flexible substrate). However, the via can additionally or alternatively be configured in any other suitable manner.

In one example overlapping stitching pattern 125b, as shown in FIG. 7A, a first conductive lead of the set of conductive leads 120 has a first portion 23a, at the first broad surface, and a second conductive lead of the set of conductive leads has a second portion 23b, at the second broad surface, wherein the first portion 23a and the second portion 23b can cross each other such that a projection of the first portion 23a onto a plane intersects a projection of the second portion 23b onto the plane. In particular, in this example, upon coupling of the first bonding layer 130 and the second bonding layer 140 to the flexible substrate no, portions of the conductive leads are insulated from each other and can be routed in a crossing stitching pattern in a manner that can reduce electromagnetic interference. In a variation of this example, portions of the set of conductive leads can additionally or alternatively be routed in a cross stitching pattern at the same broad surface of the flexible substrate no, in particular, in variations wherein the conductive leads are insulated from each other (e.g., with a non-conductive material encapsulating each of the set of conductive leads). Such a configuration can reduce interference that can magnetically couple to a region in between conductive leads of the set of conductive leads, which is particularly relevant for ECG signals (due to a characteristic lower frequency signal component for ECG signals). However, variations of the example can alternatively be configured in any other suitable manner.

In another example overlapping stitching pattern 125c, as shown in FIG. 7B, a first conductive lead of the set of conductive leads 120 has a first portion 23a, at the first broad surface, and a second conductive lead of the set of conductive leads has a second portion 23b, at the second broad surface, wherein the first portion 23a and the second portion 23b can overlap with each other such that a projection of the first portion 23a onto a plane is parallel with (i.e., does not intersect with) a projection of the second portion 23b onto the plane. In particular, in this example, the overlapping stitching pattern 125c significantly increases a distance between adjacent portions of a conductive lead on the same side of substrate no. Increasing the distance lowers the risk of portions of the set of conductive leads 120 electrically connecting to each other (e.g., from thread fraying) in an undesired manner. In addition, increasing the distance also increases manufacturing tolerances related to the positioning of a conductive lead of the set of conductive leads 120. Similar to the example described above, the first portion 23a and the second portion 23b can alternatively be configured at the same broad surface of the flexible substrate, in variations wherein the set of conductive leads 120 comprises insulated conductive leads. The set of conductive leads can, however, have any other suitable stitching configuration.

While the first variation and the second variation are described separately above, one or more portions of a conductive lead and/or of the set of conductive leads 120 can include both the first variation (i.e., first region and second region at the same side of the flexible substrate 110) and the second variation (i.e., first region and second region at opposite sides of the flexible substrate no) of the configurations described. As such, the first region 121, the second region 122, and the intermediate region 123 of a conductive lead can all be positioned at the same side of the flexible substrate. Alternatively, the intermediate region 123 of the conductive lead can cross the thickness of the flexible substrate 110 one or more times in connecting the first region 121 to the second region 122 of the conductive lead.

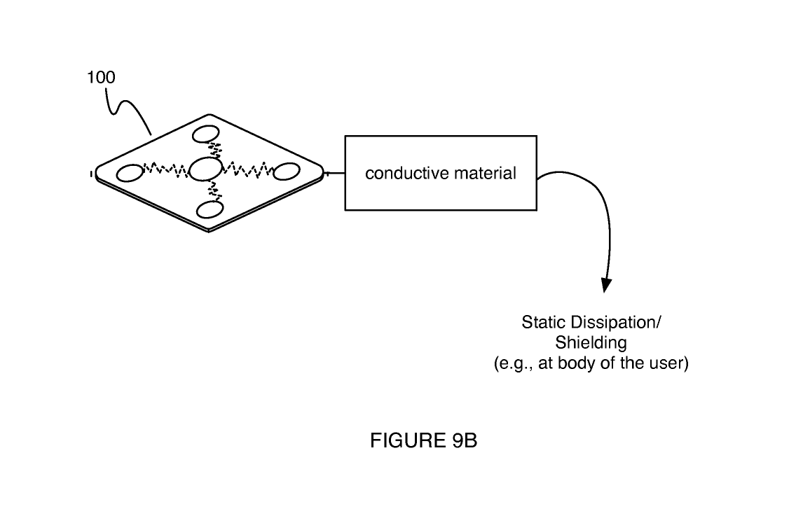

In relation to coupling between the flexible substrate 110 and the set of biosensing contacts 500 at positions proximal the set conductive leads 120, the substrate 110 can include one or more features that enhance coupling to the set of biosensing contacts 500. In a first variation, as shown in FIG. 8, the flexible substrate no can include a plurality of openings 116, proximal the set of conductive leads 120, configured to provide additional surface area to increase the peel strength between the set of biosensing contacts 500 and the flexible substrate no. Additionally or alternatively, the plurality of openings 116 in the flexible substrate 110 can provide bonding points between the substrate no and the garment 400, as described in relation to the bonding layers 130, 140 of Section 1.2.3 below. In particular, when bonding the flexible substrate 110 to the garment 400, material of a bonding layer 130, 140 can flow through the plurality of openings 116 in the flexible substrate 110 and strengthen a bond between the flexible substrate no and the garment 400. Additionally, the plurality of openings 116 can increase flexibility of the substrate 110 in response to bending and/or torsional stresses experienced during use.

Additionally or alternatively, in a second variation, the flexible substrate no can comprise a set of recesses in order to provide additional surface area to increase the peel strength between the set of biosensing contacts 500 and the flexible substrate no. Additionally or alternatively, in a third variation, the flexible substrate no can comprise an abraded surface 111 order to provide additional surface area to increase the peel strength between the set of biosensing contacts 500 and the flexible substrate no. Additionally or alternatively, in a fourth variation, an adhesive primer can be applied to a surface of the flexible substrate 110 prior to coupling of the set of biosensing contacts 500 to the flexible substrate. The regions of the flexible substrate no proximal the set of conductive leads 120 can, however, be configured in any other suitable manner to facilitate coupling between set of conductive leads 120 and the set of biosensing contacts 500.

Furthermore, in some variations, the stretching capacity of the system 100 can be increased further by making cutouts in areas of the flexible substrate 110 away from the set of conductive leads 120. As such, in one variation, the material of the set of conductive leads 120 can be coupled to the flexible substrate no in a manner that significantly reduces the area of the flexible substrate 110 coupled to the set of conductive leads 120. The set of conductive leads 120 and/or the flexible substrate 110 can, however, be processed in any other suitable manner (e.g., with electrical insulation of the set of conductive leads by a non-conductive coating) to increase stability and usability of the system 100.

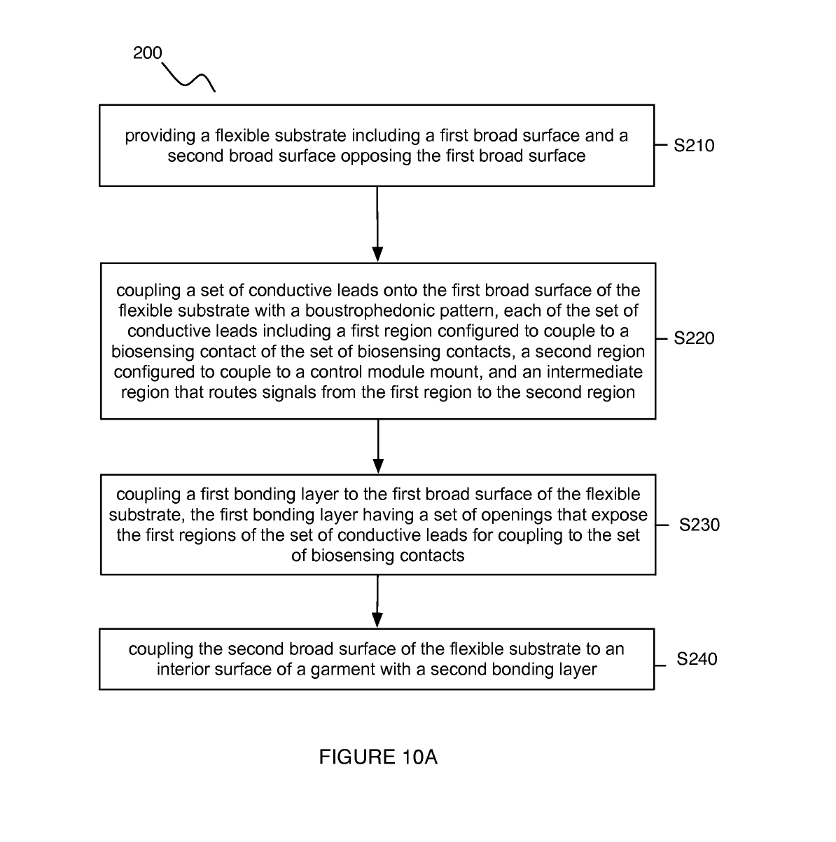

In a specific configuration of the set of conductive leads 120, as shown in FIG. 9A, a stitching pattern 125 provides multiple subsets of three conductive leads, wherein each subset of three conductive leads terminates at a sensor site. The stitching pattern 125 shown in FIG. 9A is configured for use with a short or pant garment, and includes twelve sensor sites: one sensor site for each quadriceps muscle group, one sensor site for each hamstring muscle group, one sensor site for each gluteus muscle group, and four sensors used for cardiac parameter signal detection. However, the set of conductive leads 120 can alternatively have any other suitable configuration in relation to the type of garment 400 and/or fit of the garment 400 to the user.

### 1.2.3 System—Bonding Layers

The first bonding layer 130 is coupled to the first broad surface 111 of the flexible substrate no and includes a set of openings 135 that expose the first regions of the set of conductive leads 120 for coupling to the set of biosensing contacts 500. The first bonding layer 130 is configured to couple to at least a portion of the second bonding layer 140 (described in further detail below), such that the flexible substrate no is sealed between the first bonding layer 130 and the second bonding layer 140. As such, in this variation, the set of openings 135 in the first bonding layer 130 can provide access to the set of conductive leads 120 of the substrate 110, when the material of the set of biosensing contacts 500 is coupled to the flexible substrate 110 and the set of conductive leads 120. The first bonding layer 130 can additionally function to retain the first regions 121 of the set of conductive leads 120 in position for purposes of manufacturing, wherein a first region 121 of a conductive lead is retained by one or more edges of an opening of the set of openings 135. The openings of the set of openings 135 are preferably geometrically similar to corresponding biosensing contacts of the set of biosensing contacts 500, such that coupling of the set of biosensing contacts 500 to the set of conductive leads 120 by way of the set of openings 135 forms a water tight seal that prevents moisture from damaging the system 100. However, the openings of the set of openings 135 can alternatively be geometrically dissimilar (e.g., in size, in morphology) to corresponding biosensing contacts of the set of biosensing contacts 500.

The first bonding layer 130 is preferably composed of a hydrophobic material that is impermeable to fluids; however, the material of the first bonding layer 130 can alternatively be non-hydrophobic and/or breathable while still being impermeable to fluids. A breathable material that is impermeable to fluids would prevent moisture damage, while also enhancing comfort for the user during use of the system 100. Furthermore, the material of the first bonding layer 130 can be selected to modulate stretching capability of the system 100. In variations, the first bonding layer 130 is composed of a heat-activated adhesive polymer material; however, the first bonding layer 130 can alternatively be composed of any other suitable material. In a specific example, the first bonding layer 130 comprises a polyurethane film that can be thermally bonded to the second bonding layer 140 and/or other elements of the system 100.

In variations of the first bonding layer 130 involving a polymer material, the first bonding layer 130 can be formed with varying levels of conductivity by implementing additives (e.g., of different types, in different concentrations). In variations, conductive additives including one or more of: carbon, carbon nanotubes, pellectron, lithium ion salt, and any other suitable additive may be added in various concentrations to a polymer-based resin to create a bonding layer with desired resistance properties. By controlling an amount of conductive additives, the bonding layer can additionally prevent and/or dissipate static interference, shield the conductors 120 of the flexible substrate from noise, and/or route electrical information and power through the bonding layer.

In one such variation an anti-static or static dissipating grade material can be formed similarly to as described for the flexible substrate 110 above. The anti-static properties can minimize signal interference (e.g., triboelectric effect) that could otherwise result from bending and/or stretching of the bonding layer 130 or movement and rubbing of the skin against bonding layer 130 or movement and rubbing of any other material against bonding layer 130 creating a separation of charges or static. The surface resistance of an anti-static or static dissipating material can be between 10⁶ and 10¹² ohm per square. However, the surface resistance of the bonding layer 130 can be controlled to any other suitable resistance grade.

In another such variation, the first bonding layer 130 can be configured with one or more regions having high conductivity. High conductivity regions can be used to route electrical information and power through bonding layer 130. Using this approach, a multilayered first bonding layer 130 can be formed where regions of high conductivity are separated in the “z-axis” (in the orientation shown in FIGS. 6A and 6B) by regions of low conductivity. In this variation, conductive channels or ports can be created to connect regions of high conductivity, thereby providing flexibility in the design of routing electrical signals and power through the first bonding layer 130. As an example, high conductivity regions could be formed from materials with surface resistances less than 10⁶ ohm per square and low conductivity regions from materials with surface resistances greater than 10⁶ ohm per square.

In another such variation, regions of high conductivity in bonding layer 130 can be used to electrically shield the conducting elements of the flexible substrate no. The high conductive region(s) of bonding layer 130 can form a plane parallel to and comprising a region where the conductors 120 are routed through the flexible substrate no. However, the conductive region of bonding layer 130 can be separated in the “z-axis” (in the orientation shown in FIGS. 6A and 6B) by regions of low conductivity from the conducting elements 120 of the flexible substrate, and therefore not considered to be in electrical contact with the conductors 120.

Additionally or alternatively, the high or low conductive regions of bonding layer 130 as described above can terminate at the body of the user for static or noise dissipation. A contact similar to 500 connected to the conductive regions can provide the termination of static/noise onto the body of the user. Additionally or alternatively, the system 100 can include a reference region configured to facilitate dissipation of static, as described in U.S. application Ser. No. 14/699,730 entitled “Biometric Electrode System and Method of Manufacture” and filed on 29 Apr. 2015.

However, variations of the first bonding layer 130 can be composed of any other suitable material (e.g., polymeric material) that is bondable to other elements of the system 100, in any other suitable manner (e.g., by adhesive bonding, etc.) and/or any other suitable configuration. Furthermore, in order to enhance the strength of bonding between the first bonding layer 130 and the second bonding layer 140, the first and the second bonding layers 130, 140 are preferably composed of identical materials; however, in alternative variations, the first and the second bonding layers 130, 140 can alternatively be composed of different materials.

The second bonding layer 140 is coupled to the second broad surface 112 of the flexible substrate 110 and configured to couple the flexible substrate 110 to a garment 400, as described above. The second bonding layer 140 functions to cooperate with the first bonding layer 130 to form a bonding assembly that seals sensitive portions of the flexible substrate 110 and the set of conductive leads 120 from damage or shorting that could otherwise result from fluid reaching the flexible substrate 110. Additionally or alternatively, in some variations, the second bonding layer 140 can include at least one opening 145 configured to interface with a control module mount 300 configured to receive a portable control module 30 for reception of signals from the set of biosensing contacts 500.

Similar to the first bonding layer 130, the second bonding layer 140 is preferably composed of a hydrophobic material that is impermeable to fluids; however, the material of the second bonding layer 140 can alternatively be non-hydrophobic and/or breathable, while still being impermeable to fluids. A breathable material that is impermeable to fluids would prevent moisture damage, while also enhancing comfort for the user during use of the system 100. Furthermore, the material of the second bonding layer 140 can be selected to modulate stretching capability of the system 100. In variations, the second bonding layer 140 is composed of a heat-activated adhesive polymer material; however, the second bonding layer 140 can alternatively be composed of any other suitable material. In a specific example, the second bonding layer 140 comprises a polyurethane film that can be thermally bonded to the first bonding layer 130 and/or other elements of the system 100.

Similar to the first bonding layer 130, in variations of the second bonding layer 140 involving a polymer material, the second bonding layer 140 can be formed with varying levels of conductivity by implementing additives (e.g., of different types, in different concentrations). In variations, conductive additives including one or more of: carbon, carbon nanotubes, pellectron, lithium ion salt, and any other suitable additive may be added in various concentrations to a polymer-based resin to create a bonding layer with desired resistance properties. By controlling the amount of conductive additives, the bonding layer can additionally prevent and/or dissipate static interference, shield the conductors 120 of the flexible substrate from noise, and/or route electrical information and power through the bonding layer.

In one such variation, an anti-static or static dissipating grade material can be formed as described for the first bonding layer 130 above. The anti-static properties can minimize signal interference (e.g., triboelectric effect) that could otherwise result from bending and/or stretching of the second bonding layer 140 or from rubbing and movement of the fabric of garment 400 against the second bonding layer or from rubbing of fabric layers of an outer garment worn on top of garment 400 or movement and rubbing of any other material against bonding layer 140 creating a separation of charges or static. The surface resistance of an anti-static or static dissipating material can be between 10⁶ and 10¹² ohm per square. However, the surface resistance of the second bonding layer 140 can be controlled to any other suitable resistance grade.

In another such variation, the second bonding layer 140 can be configured with one or more regions having high conductivity and one or more regions having low conductivity. High conductivity regions can be used to route electrical information and power through the low conductivity regions of the second bonding layer 140. Using this approach, a multilayered second bonding layer 140 can be formed where regions of high conductivity are separated in the “z-axis” (in the orientation shown in FIGS. 6A and 6B) by regions of low conductivity. In this variation, conductive channels or ports can be created to connect regions of high conductivity, thereby providing flexibility in the design of routing electrical signals and power through the second bonding layer 140. As an example, high conductivity regions could be formed from materials with surface resistances less than 10⁶ ohm per square and low conductivity regions from materials with surface resistances greater than 10⁶ ohm per square.

In another such variation, regions of high conductivity in bonding layer 140 can be used to electrically shield the conducting elements of the flexible substrate no. The high conductive region(s) of bonding layer 140 can form a plane parallel to and comprising a region where the conductors 120 are routed through the flexible substrate 110. However, the conductive region(s) of bonding layer 140 can be separated in the “z-axis” (in the orientation shown in FIGS. 6A and 6B) by regions of low conductivity from the conductors 120 of the flexible substrate and therefore not in electrical contact with the conductors 120.

Furthermore, in relation to the port(s) 127 through the flexible substrate no described above, one or more ports 127a, as shown in FIG. 6B, can be configured to facilitate coupling between the first bonding layer 130 and the second bonding layer 140. In some variations, high conductivity, low conductivity, and/or anti-static regions of bonding layers 130 and 140 can be connected through one or more port(s) 127. The connection through port(s) 127 couples the regions of the two bonding layers together into a single region surrounding the conductors 120 of the flexible substrate no wherein, due to contact between the first bonding layer 130 and the body of the user, the regions of bonding layers 130 and 140 are connected together and to the body of the user. By coupling bonding layers 130 and 140 through port(s) 127 a coupled area can be formed that encapsulates the conductors 120 of the flexible substrate no. Encapsulating the conductors 120 can provide noise and static shielding for the conductors 120. Additionally or alternatively, the system 100 can include a reference region configured to facilitate dissipation of static, as described in U.S. application Ser. No. 14/699,730 entitled “Biometric Electrode System and Method of Manufacture” and filed on 29 Apr. 2015.

The system 100 can include any other suitable elements configured to enhance coupling of electrode elements to a body region of a user, to dissipate static, to shield the conductors from noise, to prevent moisture damage to elements of the system 100, and/or to facilitate manufacturing of the system 100. Furthermore, as a person skilled in the art will recognize from the previous detailed description and from the figures, modifications and changes can be made to the electrode system 100 without departing from the scope of the electrode system 100.

### 2. Method of Manufacture

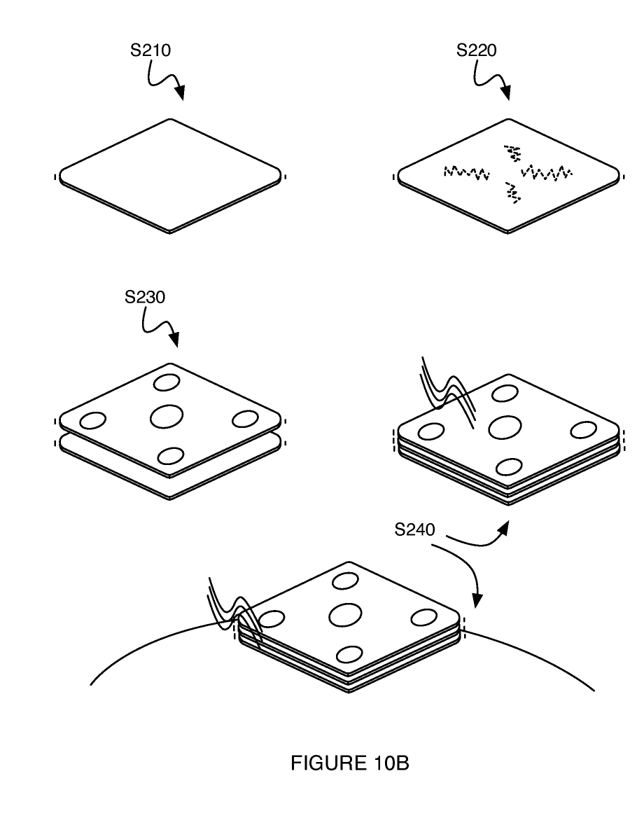

As shown in FIGS. 10A and 10B, an embodiment of a method 200 for manufacturing an electrode system comprises: providing a flexible substrate including a first broad surface and a second broad surface opposing the first broad surface S210; embroidering a set of conductive leads onto the first broad surface of the flexible substrate with a boustrophedonic pattern S220, each of the set of conductive leads including a first region configured to couple to a biosensing contact of the set of biosensing contacts, a second region configured to couple to a control module mount, and an intermediate region that routes signals from the first region to the second region; coupling a first bonding layer to the first broad surface of the flexible substrate, the first bonding layer having a set of openings that expose the first regions of the set of conductive leads for coupling to the set of biosensing contacts S230; and coupling the second broad surface of the flexible substrate to an interior surface of a garment with a second bonding layer S240.

The method 200 functions to produce an information transfer inlay system that is coupleable to a garment intended to be worn by a user while the user performs a physical activity. In particular, the method 200 functions to produce a system that is resistant to damage by fluid associated with an activity performed by an individual, and that maintains contact with the user as the user performs the activity. As such, the method 200 can provide a system configured to facilitate signal transmission associated with one or more of: electromyography (EMG) signals, electrocardiography (ECG) signals, electroencephalograph (EEG) signals, galvanic skin response (GSR) signals, bioelectric impedance (BIA) and any other suitable biopotential signal of the user. The method 200 is preferably configured to produce an embodiment, variation, or example of the system 100 described in Section 1 above; however, in other embodiments, sub-portions of the method 200 can be adapted to manufacturing portions of any other suitable system.

Block S210 recites: providing a flexible substrate including a first broad surface and a second broad surface opposing the first broad surface, which functions to provide a first portion of the system that provides coupling regions for a set of conductive leads and ultimately, a set of biosensing contacts coupled to the set of conductive leads. In embodiments, variations, and examples, the flexible substrate is preferably the flexible substrate described in Section 1.2.1 above; however, in other variations, the substrate can comprise any other suitable substrate to which the set of conductive leads and a set of biosensing contacts can be coupled.

Block S220 recites: coupling a set of conductive leads onto the first and the second broad surfaces of the flexible substrate with a boustrophedonic pattern, which functions to provide signal routing pathways from a set of biosensing contacts to a control module mount (for downstream processing of signals from the set of biosensing contacts). As noted above in Section 1.2.2, each of the set of conductive leads preferably includes a first region configured to couple to a biosensing contact of the set of biosensing contacts, a second region configured to couple to a control module mount, and an intermediate region that routes signals from the first region to the second region and through the thickness of the flexible substrate, which is described further below; however, the set of conductive leads can alternatively have any other suitable type or number of regions.

One variation of Block S220 can comprise using a needle-bobbin assembly for coupling conductive thread to the flexible substrate, wherein the needle provides a first thread and the bobbin provides a second thread. In this variation, the needle can be configured to pass from the first broad surface of the flexible substrate to the second broad surface of the flexible substrate, thereby interlocking the first thread with the second thread at the bobbin, which is located at the second broad surface of the flexible substrate. In addition, an embroidery machine comprising the needle-bobbin assembly can include multiple needles and can be fixed with an automatic bobbin changer giving the embroidery machine access to multiple bobbins that can be automatically changed.

In one such example of the variation described above, as shown in FIGURE ii, Block S220 is implemented using an embroidery machine including at least two needles and two bobbins. In this example, Block S220 comprises embroidering the conductive thread onto the first broad surface of the flexible substrate, wherein the conductive thread is used on a first needle of the embroidery machine, and a non-conductive holding thread is used on a first bobbin of the embroidery machine S222. In this example, a second bobbin holding additional conductive thread can replace the first bobbin while the conductive thread is still run through the flexible substrate with the first needle S224, to generate the set of conductive leads at the flexible substrate. As such, this example of Block S220 provides an electrical connection between the conductive thread from the first needle and the conductive thread from the second bobbin, and provides an automated embroidery approach to pass the conductive thread through the flexible substrate in generating the set of conductive leads at the first broad surface of the flexible substrate, and through to the second broad surface of the flexible substrate. Then, in this example, a second needle configured with additional non-conductive thread replaces the first needle, and the second bobbin in combination with the second needle allow the conductive thread to continue to be embroidered on the second broad surface of the flexible substrate S226. Variations of the specific example can, however, involve embroidery of the conductive thread of the set of conductive leads at the first broad surface and/or the second broad surface of the flexible substrate in any other suitable manner.

While Block S220 above describes embroidering the set of conductive leads onto the flexible substrate, variations of Block S220 can additionally or alternatively comprise coupling the set of conductive leads to the flexible substrate in any other suitable manner (e.g., using a printing method, using a molding process, using a thermal forming process, using a bonding method, using a wire-routing method, using an adhesive method, using other stitching methods, etc.).

For instance, in some variations, at least a portion of the set of conductive leads can be provided at a surface of the flexible substrate instead using a conductive polymer printed or deposited onto the flexible substrate or directly onto another layer of the system in communication with the fabric substrate (e.g., static dissipating layer, insulating layer, etc.). In particular, in one variation a pattern of conductive polymer material (e.g., conductive silicone, conductive polymer, etc.) can be coupled to the flexible substrate. In this variation, a bulk portion of the flexible substrate can be made from material that is anti-static and that has low conductivity; however, regions of the flexible substrate can include defined areas of high conductivity that allow electrical signals to pass along and/or through the flexible substrate in a desired manner. As such, in this variation of Block S220, areas of higher conductivity are coupled to regions of the flexible substrate in strategic locations to allow signals to be transmitted along conductive paths at either or both of the first broad surface and the second broad surface of the flexible substrate, and to a control module mount for transmission to a portable control module.

In any of the variations of Block S220 described above, Block S220 can additionally or alternatively include forming a conductive channel through the flexible substrate (e.g., through the thickness of the flexible substrate, through an sub-surface portion of the flexible substrate, etc.). Block S222 can include providing a conductive material within at least a portion of the flexible substrate. For example, a channel of conductive material (e.g., silicone, polymer) can be deposited (e.g., injected, printed, impregnated, etc.) within the flexible substrate at one or more locations to provide conductive ports that allow a signal to conduct through the flexible substrate (e.g., through the thickness of the flexible substrate, into a sub-surface portion of the flexible substrate, etc.). In addition, the channel of conductive material can include properties that allow for conduction in only desired directions. As such, Block S220 can comprise coupling at least a portion of a conductive lead to a conductive port produced by generating a channel of conductive material through the flexible substrate. However, regions of desired conductivity along and/or through the flexible substrate can additionally or alternatively be generated in any other suitable manner.

Block S230 recites: coupling a first bonding layer to the first broad surface of the flexible substrate, the first bonding layer having a set of openings that expose the first regions of the set of conductive leads for coupling to the set of biosensing contacts. Block S230 functions to form a portion of a bonding region that seals (e.g., in a waterproof manner) sensitive portions of the flexible substrate and protects the flexible substrate from moisture damage. In Block S230, each of the set of conductive leads includes a first region configured to couple to a biosensing contact of the set of biosensing contacts, a second region configured to couple to a control module mount, and an intermediate region that routes signals from the first region to the second region, in isolation from signals of other biosensing contacts of the set of biosensing contacts, during use by a user, as described in Section 1.2.2 above.

In Block S230, the first bonding layer is preferably composed of a hydrophobic material that is impermeable to fluids, variations of which are described in Section 1.2.3 above; however, the material of the first bonding layer used in Block S230 can alternatively be non-hydrophobic while still being impermeable to fluids. Furthermore, in order to enhance the strength of bonding between the first bonding layer of Block S230 and the second bonding layer of Block S240, the first and the second bonding layers are preferably composed of identical materials; however, in alternative variations, the first and the second bonding layers can alternatively be composed of different materials.

In this variation, Block S230 can further comprise cutting (e.g., punching, laser cutting, cutting, etc.) a set of openings (i.e., corresponding to the set of biosensing contacts) through first bonding layer and the flexible substrate thereby providing a set of openings that correspond to positions of the first regions of the set of conductive leads. As such, the set of openings can enable positioning of material of the set of biosensing contacts of the system proximal the set of conductive leads for signal transmission. As described above, one or more edges of the set of openings can additionally facilitate retention of the first regions of the set of conductive leads in position. However, variations of Block S230 can comprise forming a set of openings and/or coupling a first bonding layer composed of any other suitable material (e.g., polymeric material) to the flexible substrate in any other suitable manner (e.g., by adhesive bonding, etc.).

Block S240 recites: coupling the second broad surface of the flexible substrate to an interior surface of a garment with a second bonding layer, which functions to couple the flexible substrate to fabric of the garment. Block S240 can further function to form a second portion of a bonding region that seals sensitive portions of the flexible substrate and protects the flexible substrate from moisture. In Block S240, at least one of the set of conductive leads crosses a seam of a garment upon coupling the second broad surface to the garment with the second bonding layer. Furthermore, in Block S240, each of the set of conductive leads is preferably sealed between the first bonding layer and the second bonding layer in a waterproof manner (or even further, with a hermetic seal).

In Block S240, the second bonding layer is preferably composed of a hydrophobic material that is impermeable to fluids, as described in Section 1.2.3 above; however, the material of the second bonding layer used in Block S240 can alternatively be non-hydrophobic while still being impermeable to fluids. Variations of Block S240 can comprise coupling a second bonding layer composed of any other suitable material (e.g., polymeric material) that is bondable to other elements of the system in any other suitable manner (e.g., by adhesive bonding, etc.). Furthermore, in order to enhance the strength of bonding between the first bonding layer of Block S230 and the second bonding layer of Block S240, the first and the second bonding layers are preferably composed of identical materials; however, in alternative variations, the first and the second bonding layers can alternatively be composed of different materials.

Blocks S210-S240 can include simultaneous implementation of Blocks. Furthermore, Blocks S210-S240 can be performed in any suitable order. For instance, in one such variation, Blocks S230 and S240 can be performed simultaneously, in coupling both bonding layers to the flexible substrate (e.g., using a thermal bonding process after the layers of the system are aligned). Variations of Blocks S210-S240 can, however, be implemented in any other suitable manner.

Embodiments, variations, and examples of the method 200 can thus generate an electrode system that is thinner, lighter, and resource efficient, using a process that is less labor-intensive.

Variations of the system 100 and method 200 include any combination or permutation of the described components and processes. Furthermore, various processes of the preferred method can be embodied and/or implemented at least in part as a machine configured to receive a computer-readable medium storing computer-readable instructions. The instructions are preferably executed by computer-executable components preferably integrated with a system and one or more portions of the control module 155 and/or a processor. The computer-readable medium can be stored on any suitable computer readable media such as RAMs, ROMs, flash memory, EEPROMs, optical devices (CD or DVD), hard drives, floppy drives, or any suitable device. The computer-executable component is preferably a general or application specific processor, but any suitable dedicated hardware device or hardware/firmware combination device can additionally or alternatively execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

We claim:

1. A system for conducting signals from a set of biosensing contacts in communication with a user, the system comprising:
a flexible substrate including a first broad surface and a second broad surface opposing the first broad surface;
a set of conductive leads coupled to the first broad surface of the flexible substrate, each of the set of conductive leads including: a first region configured to couple to a biosensing contact at the first broad surface, a second region configured to couple to a control module mount, and an intermediate region that routes signals from the first region, through a port from the first broad surface to the second broad surface of the substrate, to the second region,
wherein each of the set of conductive leads has a boustrophedonic pattern that stretches and contracts during use of the system by the user, and
wherein at least one of the set of conductive leads crosses a seam of a garment, upon coupling of the system to the garment;
a first bonding layer coupled to the first broad surface of the substrate and including a set of openings that expose the first regions of the set of conductive leads for coupling to the set of biosensing contacts; and
a second bonding layer coupled to the second broad surface of the substrate and configured to couple the substrate to the garment.

2. The system of claim 1, wherein a first conductive lead of the set of conductive leads has a first portion, at the first broad surface, and a second conductive lead of the set of conductive leads has a second portion, at the second broad surface, and wherein the first portion and the second portion are arranged in an overlapping pattern, such that a projection of the first portion onto a plane intersects a projection of the second portion onto the plane.

3. The system of claim 1, wherein a first conductive lead of the set of conductive leads is encapsulated in non-conductive material and has a first portion, at the first broad surface, and wherein a second conductive lead of the set of conductive leads has a second portion, at the first broad surface, and wherein the first portion and the second portion are arranged in an overlapping pattern, such that a projection of the first portion onto a plane intersects a projection of the second portion onto the plane in a manner configured to reduce electromagnetic interference.

4. The system of claim 1, wherein a first conductive lead of the set of conductive leads has a first portion, at the first broad surface, and a second conductive lead of the set of conductive leads has a second portion, at the second broad surface, and wherein the first portion and the second portion are arranged in an overlapping pattern, such that a projection of the first portion onto a plane is parallel with a projection of the second portion onto the plane.

5. The system of claim 1, wherein the first regions of the set of conductive leads are retained in position at the set of openings of the first bonding layer.

6. The system of claim 2, wherein at least one of the first bonding layer and the second bonding layer has a region of high conductivity that enables signal transduction through regions of low conductivity.

7. The system of claim 1, wherein at least one of the first bonding layer, the second bonding layer, and the flexible substrate is composed of a material with conductive properties configured to mitigate effects of at least one of static buildup and noise.

8. The system of claim 7, wherein at least two of the first bonding layer, the second bonding layer, and the flexible substrate are coupled through a conductive channel.

9. The system of claim 7, wherein at least one of the first bonding layer, the second bonding layer and the flexible substrate is coupled to a body region of the user during use.

10. The system of claim 1, wherein the first bonding layer and the second bonding layer provide a waterproof seal about each of the set of conductive leads, and wherein at least one of the first bonding layer and the second bonding layer isolates each of the set of conductive leads from other conductive leads in the set of conductive leads.

11. A system for conducting signals from a set of biosensing contacts, the system comprising:
a flexible substrate including a first broad surface and a second broad surface opposing the first broad surface;
a set of conductive leads coupled to the first broad surface of the flexible substrate, each of the set of conductive leads including a first region configured to couple to a biosensing contact, a second region configured to couple to a control module mount, and an intermediate region that routes signals from the first region to the second region during use by a user;
a first bonding layer coupled to the first broad surface of the flexible substrate and including a set of openings that expose the first regions of the set of conductive leads for coupling to the set of biosensing contacts; and
a second bonding layer coupled to the second broad surface of the flexible substrate and configured to couple the flexible substrate to the garment.

12. The system of claim 11, wherein each of the set of conductive leads has a boustrophedonic pattern that stretches and contracts during use of the system by the user.

13. The system of claim 11, wherein at least one of the set of conductive leads crosses a seam of the garment, upon coupling of the system to the garment.

14. The system of claim 11, wherein the first bonding layer and the second bonding layer provide a waterproof seal about each of the set of conductive leads, and wherein at least one of the first bonding layer and the second bonding layer isolates each of the set of conductive leads from other conductive leads in the set of conductive leads.

15. The system of claim 11, wherein the flexible substrate includes a conductive channel into a sub-surface portion of the flexible substrate, wherein the conductive channel is coupled to the first region and the second region of at least one of the set of conductive leads.

16. The system of claim 15, wherein the intermediate region routes signals from the first region, through a port from the first broad surface to the second broad surface of the substrate, to the second region, thereby enabling signal transmission from the first broad surface and through to the second broad surface of the flexible substrate.

17. The system of claim 16, wherein a first conductive lead of the set of conductive leads has a first portion, at the first broad surface, and a second conductive lead of the set of conductive leads has a second portion, at the second broad surface, and wherein the first portion and the second portion are arranged in an overlapping pattern, such that a projection of the first portion onto a plane intersects a projection of the second portion onto the plane.

18. A method of manufacturing the system of claim 17, comprising performing an embroidery process with an embroidery machine including a first bobbin and a first needle for providing conductive thread, wherein performing the embroidery process comprises: replacing the first bobbin with a second bobbin of the embroidery machine, the second bobbin holding additional conductive thread, while conductive thread is still run through the flexible substrate with the first needle, in order to generate the set of conductive leads at the first broad surface and the second broad surface of the flexible substrate.

19. The method of claim 18, further comprising replacing the first needle with a second needle having additional non-conductive thread, and embroidering conductive thread on the second broad surface of the flexible substrate with the second bobbin and the second needle.

20. The system of claim 11, wherein at least one of the first bonding layer, the second bonding layer, and the flexible substrate is composed of a material with conductive properties configured to mitigate effects of at least one of static buildup and noise.

21. The system of claim 20, wherein at least two of the first bonding layer, the second bonding layer, and the flexible substrate are coupled through a conductive channel.

22. The system of claim 20, wherein at least one of the first bonding layer, the second bonding layer and the flexible substrate is coupled to a body region of the user during use.

23. A system for conducting signals from a set of biosensing contacts in communication with a user, the system comprising:
a flexible substrate including a first broad surface and a second broad surface opposing the first broad surface;
a set of conductive leads coupled to the first broad surface of the flexible substrate by at least one non-conductive layer, each of the set of conductive leads including: a first region configured to couple to a biosensing contact at the first broad surface, a second region configured to couple to a control module mount, and an intermediate region that routes signals from the first region, through a port from the first broad surface to the second broad surface of the substrate, to the second region,
wherein the first regions of the set of conductive leads are exposed through at least one non-conductive layer for coupling to the set of biosensing contacts; and
wherein at least one of the set of conductive leads crosses a seam of a garment, upon coupling of the system to the garment; and
a bonding layer coupled to the second broad surface of the substrate and configured to couple the substrate to the garment.
